(19) United States
(12) Patent Application Publication
(10) Pub. No.: US 2014/0255929 A1
(43) Pub. Date: Sep. 11, 2014

(54) MOSAIC TAGS FOR LABELING TEMPLATES IN LARGE-SCALE AMPLIFICATIONS

(71) Applicant: Jianbiao Zheng, Fremont, CA (US)

(72) Inventor: Jianbiao Zheng, Fremont, CA (US)

(73) Assignee: SEQUENTA, INC., South San Francisco, CA (US)

(21) Appl. No.: 14/202,990

(22) Filed: Mar. 10, 2014

Related U.S. Application Data
(60) Provisional application No. 61/776,647, filed on Mar. 11, 2013.
(60) Provisional application No. 61/829,054, filed on May 30, 2013.

(51) Int. Cl. C12Q 1/68

(52) U.S. Cl. CPC C12Q 1/6869
USPC 435/6.11

(57) ABSTRACT

The invention relates to methods of labeling nucleic acids, such as fragments of genomic DNA, with unique sequence it referred to herein as “mosaic tag,” prior to amplification and/or sequencing. Such sequence tags are useful for identifying amplification and sequencing errors. Mosaic tags minimize sequencing and amplification artifacts due to inappropriate annealing priming, hairpin formation, or the like, that may occur with completely random sequence tags of the prior art. In one aspect, mosaic tags are sequence tags that comprise alternating constant regions and variable regions, wherein each constant region has it position in the mosaic tag and comprises a predetermined sequence of nucleotides and each variable region has a position in the mosaic tag and comprises a predetermined number of randomly selected nucleotides.

This application claims priority from U.S. provisional applications Ser. No. 61/776,647 filed 11 Mar. 2013 and Ser. No. 61/829,054 filed 30 May 2013, which applications are incorporated herein by reference in their entireties.

### BACKGROUND OF THE INVENTION

The development of high throughput, or next generation, DNA sequencing technologies has revolutionized cancer research by providing tools for measuring the genetic alterations associated with cancers with unprecedented resolution, e.g. Stratton, Science, 331: 1553-1558 (2011) Parmigiani et al. Genomics, 93(1) 17 (2009): Greenman et al, Nature, 446 (7132): 153-158 (2007); Leary et al, Science Translational Medicine, 2(20): 20ra14 (24 Feb. 2010), Although a direct role for these technologies in cancer medicine, e.g. in diagnosis, prognosis and screening, seems imminent, many challenges must be overcome before such applications are realized. For example, determining relevant cancer sequences is affected not only by the biology of a cancer, but also by the presence of normal tissue, sample preparation and handling, nucleic acid extraction, amplification techniques, and sequencing chemistries, e.g. Stratton (cited above). In particular, the relatively high level of amplification and sequencing errors makes screening and detection of rare mutations difficult, despite the huge sequencing capacity of next-generation sequencing instruments. This latter challenge has been addressed by several groups with a variety or approaches that use random sequence tags for detection and/or tracking of amplification and sequencing errors, e.g. Kinde et al, Proc. Natl. Acad. Sci., 108: 9530-9535 (2011); Schmitt et al, Proc. Natl. Acad. Sci., 109(36): 14508-14513 (2012); Casbon et al, Nucleic Acids Research, 39(12): e81 (2011); and the like. Unfortunately, the use of such random sequence tags frequently leads to significant nonspecific background amplification, particularly with increases in the nucleic acid complexity of samples and the level of multiplexing in the amplification reaction.

In view of the importance of highly multiplexed nucleic acid amplification in large scale sequencing and its medical and research applications, it would be advantageous if methods were available that overcame the limitations of employing random tags in current multiplex amplification and high throughput sequencing methodologies.

### SUMMARY OF THE INVENTION

The present invention is directed to methods for using sequence tags to improve high-throughput DNA sequencing and kits for implementing such methods. The invention is exemplified in a number of implementations and applications, some of which are summarized below and throughout the specification.

In one aspect, the invention is directed to a method having the steps of (a) preparing DNA templates from nucleic acids in a sample; (b) labeling by sampling the DNA templates to form a multiplicity tag-template conjugates, wherein substantially every DNA template of a tag-template conjugate has a unique mosaic tag comprising, alternating constant regions and variable regions, each constant region having a position in the mosaic tag and a length of from 1 to 10 nucleotides of a predetermined sequence and each variable region having a position in the tag and a length of from 1 to 10 randomly selected nucleotides, such that constant regions having the same positions have the same lengths and variable region having the same positions have the same lengths; (c) amplifying the multiplicity of tag-template conjugates; (d) generating a plurality of sequence reads for each of the amplified tag-template conjugate; and (e) determining a nucleotide sequence of each of the nucleic acids by determining a consensus nucleotide at each nucleotide position of each plurality of sequence reads having identical mosaic tags.

These above-characterized aspects, as well as other aspects, of the present invention are exemplified in a number of illustrated implementations and applications, some of which are shown in the figures and characterized in the claims section that follows. However, the above summary is not intended to describe each illustrated embodiment or every implementation of the invention.

### BRIEF DESCRIPTION OF THE DRAWINGS

The novel features of the invention are set forth with particular in the appended claims. A better understanding of the features and advantages of the present invention is obtained by reference to the following detailed description that sets forth illustrative embodiments, in which the principles of the invention are utilized, and the accompanying drawings of which:

FIGS. 1A-1B illustrate an example of labeling by sampling to attach unique sequence tags to nucleic acid molecules.

FIGS. 2A-D illustrate the steps of various of the method of the invention.

FIG. 3 illustrates the use of mosaic tag in determining the se sequence of a target polynucleotide.

### DETAILED DESCRIPTION OF THE INVENTION

The practice the present invention may employ, unless otherwise indicated, conventional techniques and descriptions molecular biology (including recombinant techniques), bioinformatics, cell biology, and biochemistry, which are within the skill of the art. Such conventional techniques include, but are not limited to, sampling and analysis of blood cells, nucleic acid sequencing and analysis, and the like. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV); PER Primer: A Laboratory Manual; and Molecular Clone: A Laboratory Manual (all from Cold Spring Harbor Laboratory Press), and the like.

The invention relates to methods of labeling nucleic acids, such as fragments of genomic DNA, with unique sequence tags, referred to herein as “mosaic tags,” prior to amplification and sequencing. Such sequence tags are useful for identifying amplification and sequencing errors. Mosaic tags minimize sequencing and amplification artifacts due to inappropriate annealing, priming, hairpin formation, or the like, that may occur with completely random sequence tags of the prior art. In one aspect, mosaic tags are sequence tags that comprise alternating constant regions and variable regions, wherein each constant region has a position in the mosaic tag and comprises a predetermined sequence of nucleotides and each variable region has a position in the mosaic tag and comprises a predetermined number of randomly selected nucleotides. By way of illustration, a 22-mer mosaic tag (SEQ ID NO: 4) may have the following form:

Nucleotide position:

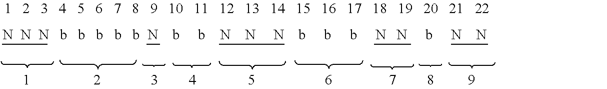

### Region Position

There are nine constant and variable regions, with regions 1 (nucleotides 1-3), 3 (nucleotide 9), 5 (nucleotides 12-14), 7 (nucleotides 18-19) and 9 (nucleotides 21-22) being variable (double underlined nucleotides) and regions 2 (nucleotides 4-8), 4 (nucleotides 10-11), 6 (nucleotides 15-17), and 8 (nucleotide 20) being constant. N represents a randomly selected nucleotide from the set of A, C, G or T; thus, the number of mosaic tags of this example is 4″=4,194,304 tags. b represents a predetermined nucleotide at the indicated position. In some embodiments, the sequence of b's, “***bbbbb*bb***bbb***b**,” is selected to minimize the likelihood of having a perfect match in a genome of the organism making up the sample.

In one aspect, for mosaic tags of a particular embodiment of the method of the invention, all constant regions with the same position have the same length and all variable regions with the same position have the same length. This allows mosaic tags to be synthesized using partial combinatorial synthesis with conventional chemistries and instruments.

In one aspect, mosaic tags comprise from 10 to 100 nucleotides, or from 12 to 80 nucleotides, or from 15 to 60 nucleotides. In some embodiments, mosaic tags comprise at least eight nucleotide positions with randomly selected nucleotides; in other embodiments, whenever mosaic tags have a length of at least 15 nucleotides, they comprise at least 12 nucleotide positions with randomly selected nucleotides. In another aspect, no variable region within a mosaic tag may have a length that is greater than seven nucleotides.

In another aspect, mosaic tags may be used in the following steps: (i) preparing DNA templates from nucleic acids in a sample; (ii) labeling by sampling the DNA templates to form a multiplicity tag-template conjugates, wherein substantially every DNA template of as tag-template conjugate has a unique mosaic tag comprising alternating constant regions and variable regions, each constant region having a position in the mosaic tag and a length of from 1 to 10 nucleotides of as predetermined sequence and each variable region having a position in the mosaic tag and a length of from 1 to 10 randomly selected nucleotides, such that constant regions having the same positions have the same lengths and variable region having the same positions have the same lengths; (iii) amplifying the multiplicity of tag-template conjugates; (iv) generating a plurality of sequence reads for each of the amplified tag-template conjugates; and (v) determining as nucleotide sequence of each of the nucleic acids by determining a consensus nucleotide at each nucleotide position of each plurality of sequence reads haying identical mosaic tags. In another aspect, mosaic tags may be used in the following steps: (a) preparing single stranded DNA templates from nucleic acids in a sample; (b) labeling by sampling the single stranded DNA templates to form tag-template conjugates, wherein substantially every single stranded DNA template of a tag-template conjugate has a unique sequence tag (that is, a mosaic tag) having a length of at least 15 nucleotides and having the following form:

[(N₁N₂ . . . N_{Kj})(b₁b₂ . . . b_{Lj})]M

wherein each Nᵢ, for i=1, 2, . . . , Kⱼ, is a nucleotide randomly selected from the group consisting of A, C, G and T; Kᵢ is an integer in the range of from 1 to 10 for each j less than or equal to M (that is, regions N₁N₂ . . . N_{Kj} are variable regions); each b₁, for i=1, 2, . . . Lⱼ, is a nucleotide; Lⱼ is an integer in the range of from 1 to 10 for each j less than or equal to M; such that every sequence tag (i) has the same Kⱼ for every j and (ii) has the same sequences b₁b₂ . . . b_{Lj} for every j (that is, regions b₁b₂ . . . b_{Lj} are constant regions); and M is an integer greater than or equal to 2; (c) amplifying the tag-template conjugates; (d) generating as plurality of sequence reads for each of the amplified tag-template conjugates; and (e) determining a nucleotide sequence of each of the nucleic acids by determining a consensus nucleotide at each nucleotide position of each plurality of sequence reads having identical sequence tags. In some embodiments, the plurality or sequence reads is at least 10⁴; in other embodiments, the plurality of sequence reads is at least 10⁵; in still other embodiments, the plurality of sequence reads is at least 10⁶. In some embodiments, the total length of the above sequence tag is in the range of from 15 to 80 nucleotides.

In one embodiment of the invention, sequence tags are attached to target nucleic acid molecules of a sample by labeling by sampling, e.g. as disclosed by Brenner et al. U.S. Pat. No. 5,846,719; Brenner et al, U.S. Pat. No. 7,537,897; Macevicz, International patent publication WO 2005/111242; and the like, which are incorporated herein by reference. In labeling by sampling, polynucleotides of a population to be labeled (or uniquely tagged) are used to sample (by attachment, linking, or the like) sequence tags of a much larger population. That is, if the population of polynucleotides has K members (including replicates of the same polynucleotide) and the population of sequence tags has N members, then N>>K. In one embodiment, the size of a population of sequence tags used with the invention is at least 10 times the size of the population of clonotypes a sample; in another embodiment, the size of a population of sequence tags used with the invention is at least 100 times the size of the population of clonotypes in a sample; and in another embodiment, the size of a population of sequence tags used with the invention is at least 1000 times the size of the population of clonotypes a sample. In other embodiments a size of sequence tag population is selected so that substantially every clonotype in a sample will have a unique sequence tag whenever such clonotypes are combined with such sequence tag population, e.g. in an attachment reaction, such as a ligation reaction, amplification reaction, or the like. In sonic embodiments, substantially every clonotype means at least 90 percent of such clonotypes will have a unique sequence tag; in other embodiments, substantially every clonotype means at least 99 percent of such clonotypes will have a unique sequence tag; in other embodiments, substantially every clonotype means at least 99.9 percent of such clonotypes will have a unique sequence tag.

In some embodiments, in which up to 1 million target nucleic acids are labeled by sampling, large sets of sequence tags may be efficiently produced by combinatorial synthesis by reacting a mixture of all four nucleotide precurors at each addition step of a synthesis reaction, e.g. as disclosed in Church, U.S. Pat. No. 5,149,625, which is incorporated by reference.

A variety of different attachment reactions be used to attach unique tags to substantially every target nucleic acid in a sample. In one embodiment, such attachment is accomplished by combining a sample containing target nucleic acid molecules with a population or library of sequence tags so that members of the two populations of molecules can randomly combine and become associated or linked, e.g. covalently. In such tag attachment reactions, target nucleic acids may comprise linear single or double stranded polynucleotides and sequence tags are carried by reagent such as amplification primers, such as PCR primers, ligation adaptors, circularizable probes, plasmids, or the like, Several such reagents capable of carrying sequence tag populations are disclosed in Macevicz, U.S. Pat. No. 8,137,936; Faham et al, U.S. Pat. No. 7,862,999; Drmanac et al, U.S. patent publication US 2009/0264299; Zheng et al, U.S. Pat. No. 7,862,999; Landegren et al, U.S. Pat. No. 8,053,188; Unrau and Deugau. Gene, 145; 163-169 (1994); Church, U.S. Pat. No. 5,149,625; and the like, which are incorporated herein by reference.

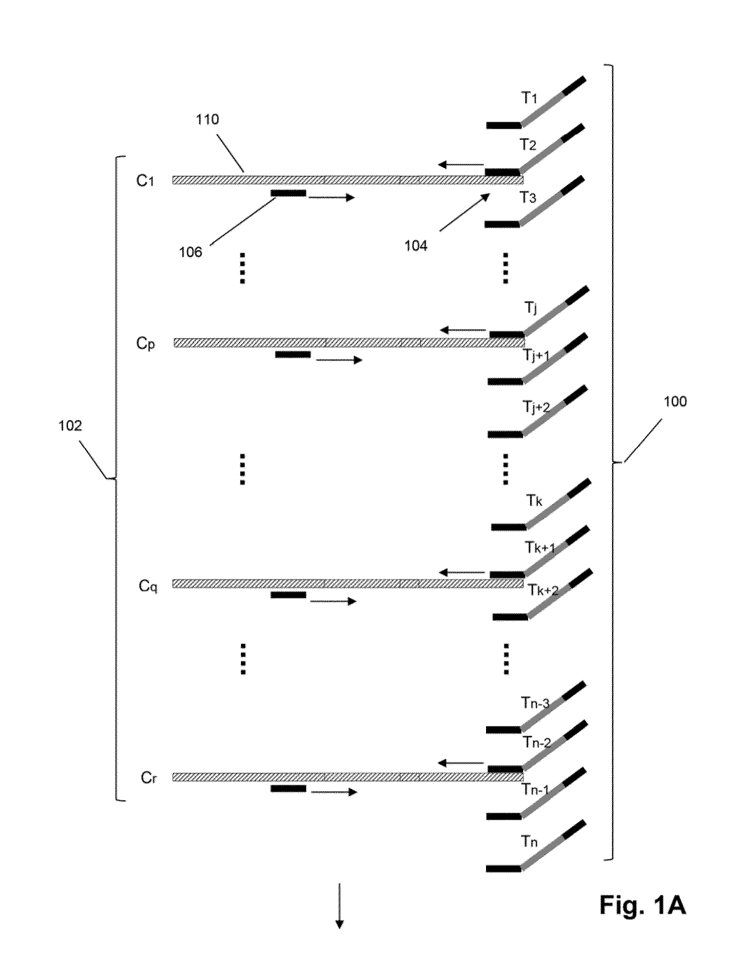

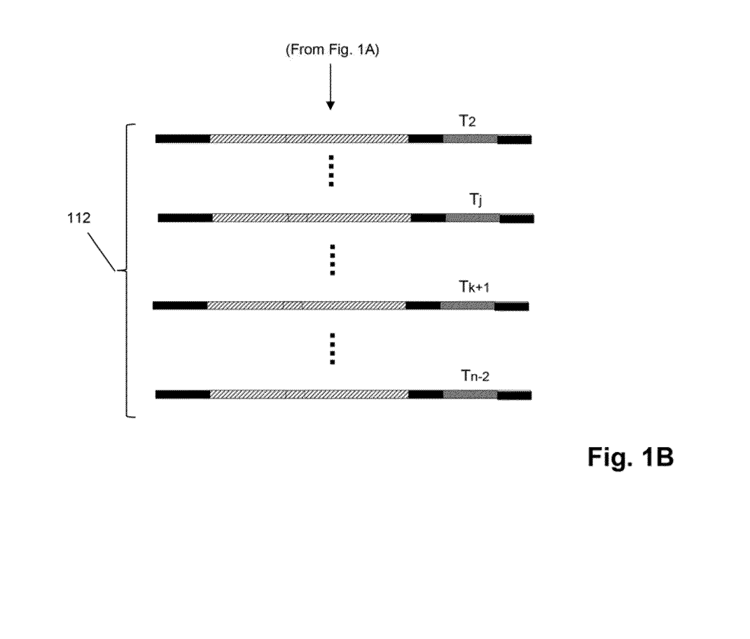

FIGS. 1A and 1B illustrate an attachment reaction comprising a reaction in which a population of sequence tags (T₁, T₂, T₃ . . . Tⱼ, Tⱼ₊₁ . . . Tₖ, Tₖ₊₁ . . . T_{B-1}, T_{g}) is incorporated into primers (100) by two or more cycles of annealing and polymerase extension, each separated by a denaturation step, The population of sequence tags has a ranch greater size than that of target nucleic acid molecules (102). The sequence tags are attached to the target nucleic acid molecules by annealing the primers to the target nucleic acid molecules and extending the primers with a DNA polymerase. The figure depicts how the target nucleic acid molecules select, or sample, a small fraction of the total population of sequence tags by randomly annealing to the primers by way of their common primer binding regions (104). Since the primers (an therefore sequence tags) combine with the target nucleic acid molecules randomly, there is only a small possibility that the same sequence tag may be attached to different nucleic acid molecules; however, if the population of sequence tags is large as taught herein, then such possibility will be negligibly small so that substantially every target nucleic acid molecule will have a unique sequence tag attached. The other primer (106) of the forward and reverse primer pair anneals to another region of the target nucleic acid (110) so that after two or more cycles of annealing, extending and melting, amplicon (112) is formed, thereby attaching unique sequence tags to each target nucleic acid (C₁, . . . Cₚ, . . . C_{q}, . . . and Cᵣ) in population (102), which may be, for example, V(D)J regions of immune receptor sequences. That is, amplicon (112) comprises the tag-template conjugates from the attachment reaction.

Such immune molecules typically form an immune repertoire which comprises a very large set of very similar polynucleotides (e.g. >1000, but more usually from 100,000 to 1,000,000, or more) which are relatively short in length (e.g. usually less than 300 bp). In one aspect of the invention, the inventors recognized and appreciated that these characteristics permitted the use of highly dissimilar sequence tags to efficiently compare sequence reads of highly similar clonotypes to determine whether they are derived from the same original sequence or not.

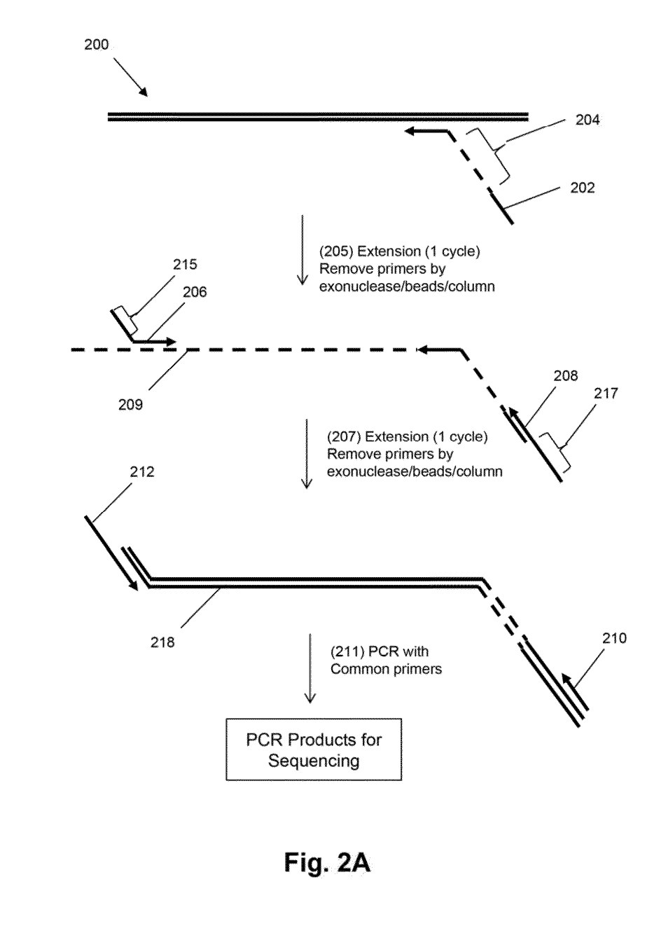

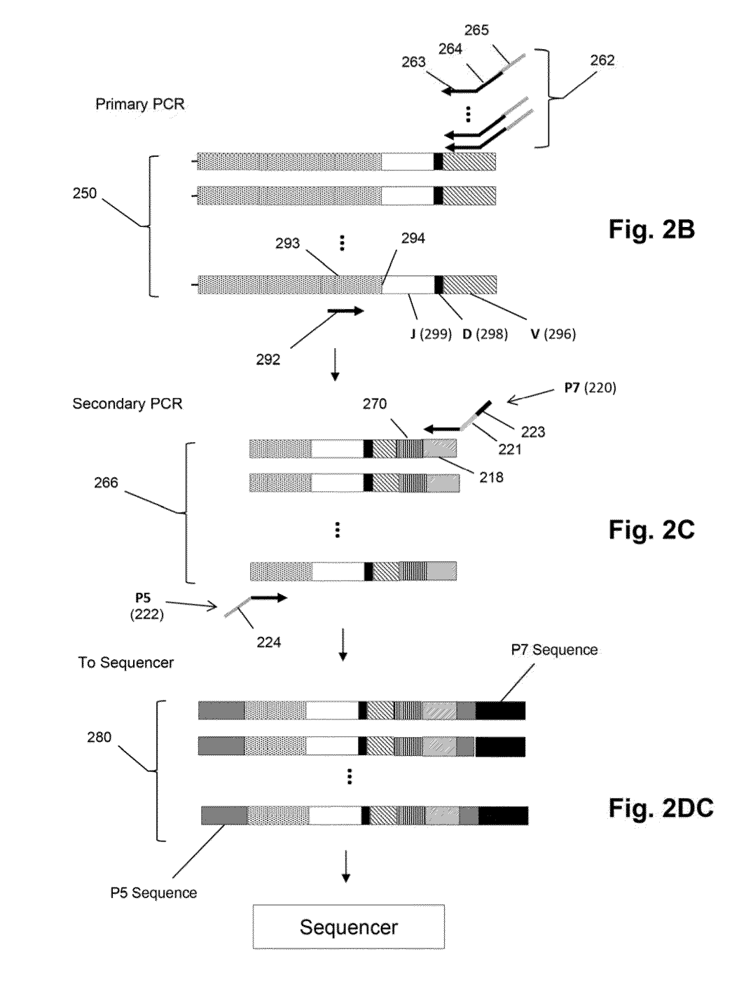

The application of mosaic tags in accordance with one aspect of the invention is illustrated in FIG. 2A. There target polynucleotide (200), which may be a fragment of genomic DNA, or the like, is combined with pruner (202) containing mosaic tag (204) in a polymerase chain reaction mixture, after which the mixture is heated to melt target polynucleotide (200) then cooled so that printer (202) cold anneal to its specific binding site and be extended (205) to form first strand (209). After melting, first stage forward primer (206) and first stage reverse primer (208) are annealed to first strand (209) and extended (207) to form fragment (218) with mosaic tag (204) embedded in it. In some embodiments, first stage primers (206) and (208) may be designed to include tails (215 and 217, respectively) that serve as primer binding sites for bridge amplification primers P5 (212) and P7 (210), for example, used with the Illumina sequencing system. Alter amplification (211) with these primers the resulting amplicons are sequenced.

FIGS. 2B-2D illustrate another set of exemplary steps of attaching unique sequence tags to recombined nucleic acid molecules in a two stage PCR. Population of recombined nucleic acid molecules (250) from a sample containing T-cells or B-cells are combined in a PCR mixture with forward and reverse primers (292) (at C region-J region boundary (294)) and (262), respectively. Reverse primers (262) each comprise three regions: target annealing region (263) (which in this illustration is V region (296); sequence tag (264); and primer binding region (265) for the second stage of the two-stage PCR. In this illustration, primers (262) comprise a mixture of target annealing regions to account for the diversity of V region sequence. Thus, every different pruner is prepared with a sequence tag region. Alternatively, the sequence tag demerit may be attached to C region pruner (292) along with a primer binding region for the second PCR stage. As noted, recombined nucleic acid molecules (250) comprise constant, or C, region (293), J region (299), D region (298), and V region (296), which may represent a V(D)J segment encoding a CDR3 region of either a TCR or immunoglobulin. After a few cycles, for example, 4 to 10, first stage amplicon (266) is produced with each member polynucleotide including sequence tag (270). In the second stage PCR, polynucleotides of amplicon (266) are reamplified with new forward and reverse primers P5 (222) and P7 (220) which add further primer binding sites (224) and (223) for cluster formation using bridge PCR in a Solexa/Illumina sequencer. Printer P7 also include a secondary sequence tag (221) for optional multiplexing of samples in a single sequencing run. After the secondary PCR amplicon (280) is produced with embedded P5 and P7 sequences by which bridge PCR may be carried out.

### Sequence Determination Using Mosaic Tags

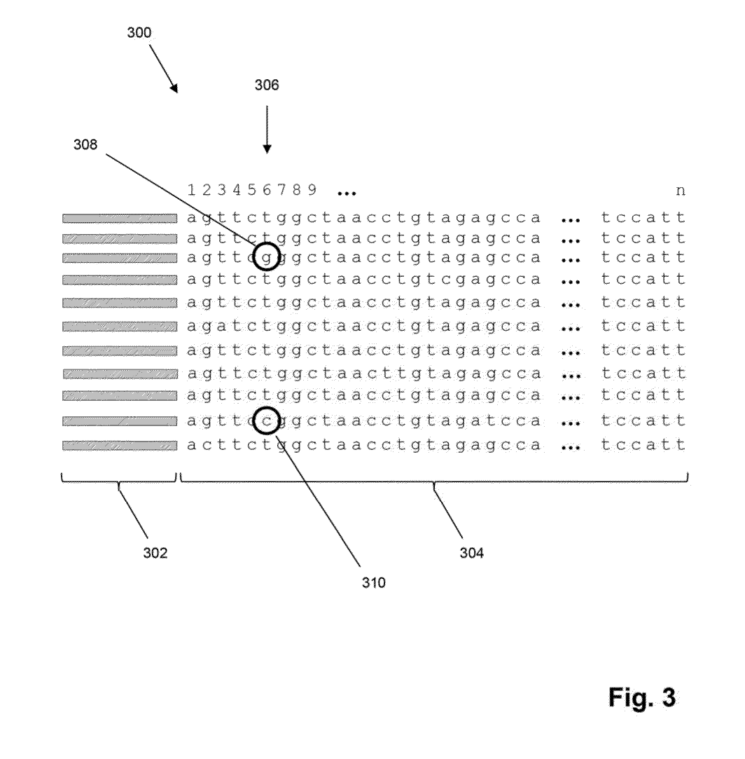

In accordance with one aspect of the invention, target polynucleotides of sample are determined by first grouping sapience reads based on their sequence tags, that is, their mosaic tags. Such grouping may be accomplished by conventional sequence alignment methods. Guidance for selecting alignment methods is available in Batzoglou, Briefings in Bioinformatics, 6: 6-22 (2005), which is incorporated by reference. After sequence reads are assembled in groups corresponding to unique sequence tags, then the sequences of the associated target polynucleotides may be analyzed to determine the sequence of the target polynucleotide from the sample. FIG. 3 illustrates an exemplary alignment and method from determining the sequence of a target polynucleotide associated with a unique mosaic tag. Sequence reads (300) pursuant each comprise a copy of a sequence tag (302) and a copy of a clonotype (304) (SEQ ID NO: 1). All sequence reads having the same sequence tag are assembled so that the nucleotides of each position in the clonotype portion can be compared (e.g., SEQ ID NO: 2 and SEQ ID NO: 3). In this example, eleven sequence reads are aligned by way of their respective mosaic tags (302) after which nucleotides at each position of the target polynucleotide portions of the sequence reads, indicated as 1, 2, 3, 4, . . . n, are compared. For example, nucleotides at position 6 (306) are t, t, g, t, t, t, t, t, t, c, t; that is, nine base calls are t's, one is “g” (308) and one is “c” (310). In one embodiment, the correct base call of the target polynucleotide sequence at a position is whatever the identity of the majority base is. In the example of position 6 (306), the base call is “t”, because it is the nucleotide in the majority of sequence reads at that position. In other embodiments, other factors may be taken into account to determine a correct base call for a target polynucleotide sequence, such as quality scores of the base calls of the sequence reads, identities of adjacent bases, or the like.

In some embodiments, mosaic tags may be used in a method of determining clonotypes of an immune repertoire comprising the steps: (a) obtaining a sample from an individual comprising T-cells and/or B-cells; (b) attaching mosaic tags to molecules of recombined nucleid acids of T-cell receptor genes or immunoglobulin genes of the T-cells and/or B-cells to form tag-molecule conjugates, wherein substantially every molecule of the tag-molecule conjugates has a unique mosaic tag; (c) amplifying the tag-molecule conjugates; (d) sequencing the tag-molecule conjugates; and (e) aligning sequence reads of like sequence tags to determine sequence reads corresponding to the same clonotypes of the repertoire. In some embodiments, said step of aligning further includes determining a nucleotide sequence of each of said clonotype of each of said tag-molecule conjugate by determining a majority nucleotide at each nucleotide position of said clonotypes of said like mosaic tags. In further embodiments, said step of attaching includes labeling by sampling said molecules of recombined nucleic acids. In further embodiments, said step of attaching is implemented in a reaction mixture such that said mosaic tags are present in the reaction mixture in a concentration at least 100 times that of said molecules of recombined nucleic acid.

In some embodiments, mosaic tags may be used method of determining a number of lymphocytes in a sample comprising the steps of: (a) obtaining a sample from an individual comprising lymphocytes; (b) attaching mosaic tags to molecules of recombined nucleic acids of T-cell receptor genes or of immunoglobulin genes of the lymphocytes to form tag-molecule conjugates, wherein substantially every molecule of the tag-molecule conjugates has a unique mosaic tag; (c) amplifying the tag-molecule conjugates; (d) sequencing the tag-molecule conjugates; (e) counting the number of distinct mosaic tags to determine the number of lymphocytes in the sample. In further embodiments, said recombined nucleic acids are DNAs. In still further embodiments, said lymphocyte is a T-cell and said recombined nucleic acids are T-cell receptor genes or fragments thereof. In still further embodiments, said lymphocyte is a B-cell and said recombined nucleic acids are immunoglobulin genes or fragments thereof.

### Kits

Some embodiments of the invention may comprise a kit for labeling each of multiple target polynucleotides with a unique sequence tag. Such kits may comprise a plurality of primers comprising at least one primer specific for each one of the multiple target polynucleotides, wherein each primer comprises a mosaic tag comprising alternating constant regions and variable regions, each constant region having a position in the mosaic tag and a length of from 1 to 10 nucleotides of a predetermined sequence and each variable region having a position in the mosaic tag and a length of from 1 to 10 randomly selected nucleotides, such that constant regions having the same positions have the same lengths and variable region having the same positions have the same lengths.

Kits may include any delivery system for delivering materials or reagents for carrying out a method of the invention. In the context of methods of the invention, such delivery systems include systems that allow for the storage, transport, or delivery of reaction reagents e.g., primers, enzymes, internal standards, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains primers.

### Sequencing Populations or Tag-Template Conjugates

Any high-throughput technique for sequencing nucleic acids can be used in the method of the invention. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, sequencing by synthesis, real time monitoring of the incorporation of labeled nucleotides during a polymerization step, polony sequencing, SOLiD sequencing, and the like. In some embodiments of the invention, high-throughput methods of sequencing are employed that comprise a step of spatially isolating individual molecules on a solid surface where they are sequenced in parallel. Such solid surfaces may include nonporous surfaces (such as in Solexa sequencing, e.g. Bentley et al, Nature, 456; 53-59(2008) or Complete Genomics sequencing, e.g. Drmanac et al, Science, 327: 78-81 (2010)), arrays of wells, which may include bead- or particle-bound templates (such as with 454, e.g. Margulies et al, Nature, 437: 376-380 (2005) or Ion Torrent sequencing, U.S. patent publication 2010/0137143 or 2010/0304982), micromachined membranes (such as with SMRT sequencing, e.g. Eid et al, Science, 323: 133-138 (2009)), or bead arrays (as with SOLiD sequencing or polony sequencing, e.g. Kim et al, Science, 316: 1481-1414 (2007)). In some embodiments, such methods comprise amplifying the isolated molecules either before or after they are spatially isolated on a solid surface. Prior amplification may comprise emulsion-based amplification, such as emulsion PCR, rolling circle amplification. Of particular interest is Solexa-based sequencing where individual template molecules are spatially isolated on a solid surface, after which they are amplified in parallel by bridge PCR to form separate clonal populations, or clusters, and then sequenced, as described in Bentley et al (cited above) and in manufacturer's instructions (e.g. TruSeq™ Sample Preparation Kit and Data Sheet, Illumina, Inc., San Diego, Calif., 2010): and further in the following references: U.S. Pat. Nos. 6,090,592; 6,300,070; 7,115,400, and EP0972081B1; which are incorporated by reference. In one embodiment, individual molecules disposed and amplified on a solid surface form clusters in a density of at least 10⁵ clusters per cm²; or in a density of at least 5×10⁵ per cm²; or in a density of at least 10⁶ clusters per cm².

The sequencing technique used in the methods of the provided invention can generate sequence reads of about 30 nucleotides, about 40 nucleotides, about 50 nucleotides, about 60 nucleotides, about 70 nucleotides, about 80 nucleotides, about 90 nucleotides, about 100 nucleotides, about 110, about 120 nucleotides per read, about 150 nucleotides, about 200 nucleotides, about 250 nucleotides, about 300 nucleotides, about 350 nucleotides, about 400 nucleotides, about 450 nucleotides, about 500 nucleotides, about 550 nucleotides, or about 600 nucleotides per read.

### EXAMPLE

Instead of a string of “N” bases, such as “. . . NNNNNNNNNNNNNNNN . . . ,” (SEQ ID NO: 5), the string of random “N” bases is broken up with insertion of specific bases (constant regions) that will minimize the interaction of molecular tags with specific oligos used (either manually or by silicon software to pick the right fixed bases). For example, the above string of N's may be broken up as follows:

. . . NNetNNtgNNgtNNgeNNtgNNgtNNtaNN . . . (SEQ ID NO: 6) The number of “N” (2 in above case) that can be placed together and the specific bases and its number used between “N” (2 in above case) will be depend on the specific oligos used. Therefore, a simple software program can be written to perform the specific function that can be used in silicon to minimize the interaction, allow the successful selection of the fixed bases. The number of “N” bases can be positioned in various different locations for the same molecular tags. For example, we can have: . . . NNctNtgNNNgtNgcNtgNNNgtNNtaNNN . . . (SEQ ID NO: 7) This approach can be applied to either single-side sequence tags or dual tags (two sequence tags attached to each target polynucleotide). The incorporation of sequence tags can be done either by extension, ligation, or combination of extension/ligation (or after flap removal).

Mosaic tags may be applied to methods of immune repertoire sequencing or rare mutation detection, for example, as disclosed in Faham and Willis, U.S. patent publication 2011/0207134; Vogelstein et al, International patent application WO/2012/142213; and the like, which are incorporated herein by reference. An example of the incorporation of mosaic molecular tags in IgH J oligo pool (see Faham and Willis, cited above) is shown in Table 1.

TABLE 1
Oligos used for IgH J pool with 15 “N” mosaic molecular tags.

| | | SEQ |
|---|---|---|
| tgHJ_15NM18 | | ID |
| primers | sequence (molecular tag/diversity: 15N) | NO: |
| IgHJ1_MoN15M18 | acg aGC ctc AtG cgT AGA NNctNtNN acNNgtNcNN | 8 |
| | acNNgtNNNctcacCTGAGGAGACGGTGA | |
| IgHJ2_MoN15M18 | acg aGC ctc AtG cgT AGA NNctNtNN acNNgtNcNN | 9 |
| | acNNgtNNNctcacCTGAGGAGACaGTGA | |
| IgHJ3_MoN15M18 | acg aGC ctc AtG cgT AGA NNctNtNN acNNgtNcNN | 10 |
| | acNNgtNNNcttacCTGAaGAGACGGTGA | |
| IgHJ6_MoN15M18 | acg aGC ctc AtG cgT AGA NNctNtNN acNNgtNcNN | 11 |
| | acNNgtNNNcttacCTGAGGAGACGGTGA | |

While the present invention has been described with reference to several particular example embodiments, those skilled in the art will recognize that many changes may be made thereto without departing from the spirit and scope of the present invention. The present invention is applicable to a variety of sensor implementations and other subject matter, in addition to those discussed above.

### Definitions

Unless otherwise specifically defined herein, terms and symbols of nucleic acid chemistry, biochemistry, genetics, and molecular biology used herein follow those of standard treatises and texts in the field, e.g. Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Abbas et al, Cellular and Molecular Immunology, 6^{th} edition (Saunders, 2007).

“Amplicon” means the product or a polynucleotide amplification reaction; that is, a clonal population of polynucleotides, which may be single stranded or double stranded, which are replicated from one or more starting sequences. The one or more starting sequences may be one or more copies of the same sequence, or they may be a mixture of different sequences. Preferably, amplicons are formed by the amplification of a single starting sequence. Amplicons way be produced by a variety of amplification reactions whose products comprise replicates of the one or more starting, or target, nucleic acids. In one aspect, amplification reactions producing amplicons are “template-driven” in that base pairing of reactants, either nucleotides or oligonucleotides, have complements in a template polynucleotide that are required for the creation of reaction products. In one aspect, template-driven reactions are primer extensions with a nucleic acid polymerase or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references that are incorporated herein by reference: Mullis et al, U.S. Pat. Nos. 4,683,195; 4,965,188; 4,683,202; 4,800,159 (PCR); Gelfand et al, U.S. Pat. No. 5,210,015 (real-time PCR with “taqman” probes); Wittwer U.S. Pat. No. 6,174,670; Kacian et al, U.S. Pat. No. 5,399,491 (“NASBA”) Lizardi, U.S. Pat. No. 5,854,033; Aono et al, Japanese patent publ. JP 4-262799 (rolling circle amplification); and the like. In one aspect, amplicons of the invention are produced by PCRs. An amplification reaction may be a “real-time” amplification if a detection chemistry is available that permits a reaction product to be measured as the amplification reaction progresses, e.g. “real-time PCR” described below, or “real-time NASBA” as described in Leone et al, Nucleic Acids Research, 26: 2150-2155 (1998), and like references. As used herein, the term “amplifying” means performing an amplification reaction. A “reaction mixture” means a solution containing all the necessary reactants for performing a reaction, which may include, but not be limited to, buffering agents to maintain pH at a selected level during a reaction, salts, co-factors, scavengers, and the like.

“Fragment”, “segment”, or “DNA segment” refers to a portion of a larger DNA polynucleotide or DNA, A polynucleotide, for example, can be broken up, or fragmented into, a plurality of segments. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range.

“Kit” refers to any delivery system for delivering materials or reagents for carrying out a method of the Invention. In the context of methods of the invention, such delivery systems include systems that allow for the storage, transport, or delivery of reaction reagents (e.g., primers, enzymes, internal standards, etc. in the appropriate containers) and/or supporting materials (e.g., buffers, written instructions for performing the assay etc.) from one location to another. For example, kits include one or more enclosures (e.g., boxes) containing the relevant reaction reagents and/or supporting materials. Such contents may be delivered to the intended recipient together or separately. For example, a first container may contain an enzyme for use in an assay, while a second container contains primers.

“Nucleic acid sequence-based amplification” or “NASBA” is an amplification reaction based on the simultaneous activity of a reverse transcriptase (usually avian myeloblastosis virus (AMV) reverse transcriptase), an RNase H, and an RNA polymerase (usually T7 RNA polymerase) that uses two oligonucleotide primers, and which under conventional conditions can amplify a target sequence by a factor in the range of 109 to 1012 in 90 to 120 minutes. In NASBA reaction, nucleic acids are a template for the amplification reaction only if they are single stranded and contain a primer binding site. Because NASBA is isothermal (usually carried out at 41° C. with the above enzymes), specific amplification of single stranded RNA may be accomplished if denaturation of double stranded DNA is prevented in the sample preparation procedure. That is, it is possible to detect a single stranded RNA target in a double stranded DNA background without getting false positive results caused by complex genomic DNA, in contrast with other techniques, such as RT-PCR. By using fluorescent indicators compatible with the reaction, such as molecular beacons. NASBAs may be carried out with real-time detection of the amplicon. Molecular beacons are stem-and-loop-structured oligonucleotides with a fluorescent label at one end and a quencher at the other end, e.g. 5′-fluorescein and 3′-(4-(dimethylamino)phenyl)lazo) benzoic acid (i.e., 3′-DABCYL), as disclosed by Tyagi and Kramer (cited above). An exemplary molecular beacon may have complementary stem strands of six nucleotides, e.g. 4 G's or C's and 2 A's or T's, and a target-specific loop of about 20 nucleotides, so that the molecular beacon can form a stable hybrid with a target sequence at reaction temperature, e.g. 41° C. A typical NASBA reaction mix is 80 mM Tris-HCl [pH 8.5], 24 mM MgCl2, 140 mM KCl, 1.0 mM DTT, 2.0 mM of each dNTP, 4.0 mM each of ATP, UTP and CTP, 3.0 mM GTP, and 1.0 mM ITP in 30% DMSO. Primer concentration is 0.1 μM and molecular beacon concentration is 40 nM. Enzyme mix is 375 sorbitol, 2.1 μg BSA, 0.08 U RNase H, 32 UT7 RNA polymerase, and 6.4 U AMV reverse transcriptase. A reaction may comprise 5 μL sample, 10 μL. NASBA reaction mix, and 5 μL enzyme mix, for a total reaction volume of 20 μL. Further guidance for carrying out real-time NASBA reactions is disclosed in the following references that are incorporated by reference: Polstra et al, BMC Infectious Diseases, 2: 18 (2002); Leone et al, Nucleic Acids Research, 26: 2150-2155 (1998); Gulliksen, et al, Anal. Chem., 76: 9-14 (2004); Weusten et al, Nucleic Acids Research, 30(6) e26 (2002); Deiman et al. Mol. Biotechnol., 20: 163-179 (2002). Nested NASBA reactions are carried out similarly to nested PCRs;. namely, the amplicon a first NASBA reaction becomes the sample fix a second NASBA reaction using a new set of primers, at least one of which binds to an interior location of the first amplicon.

“Polymerase chain reaction,” or “PCR,” means a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors well-known to those of ordinary skill the art, e.g. exemplified by the references: McPherson et al, editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature >90° C. primers annealed at a temperature in the range 50-75° C., and primers extended at a temperature in the range 72-78° C. The term “PCR” encompasses derivative forms of the reaction, including but not limited to, RT-PCR, real-time PCR, nested PCR, quantitative PCR, multiplexed PCR, and the like. The particular format of PCR being employed is discernible by one skilled in the art from the context of an application. Reaction volumes range from a few hundred nanoliters, e.g. 200 nL, to a few a hundred μL, e.g. 200 μL. “Reverse transcription PCR,” or “RT-PCR,” means a PCR that is preceded by a reverse transcription reaction that converts a target RNA to a complementary single stranded DNA, which is then amplified, e.g. Tecott et al, U.S. Pat. No. 5,168,038, which patent is incorporated herein by reference. “Real-time PCR” means a PCR for which the amount of reaction product, i.e. amplicon, is monitored as the reaction proceeds. There are many forms of real-time PCR that differ mainly in the detection chemistries used for monitoring the reaction product, e.g. Gelfand et al, U.S. Pat. No. 5,210,015 (“taqman”); Wittwer et al, U.S. Pat. Nos. 6,174,670 and 6,569,627 (intercalating dyes); Tyagi et al, U.S. Pat. No. 5,925,517 (molecular beacons); which patents are incorporated herein by reference. Detection chemistries for real-time PCR are reviewed in Mackay et al, Nucleic Acids Research, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out the same reaction mixture, e.g. Bernard et al, Anal. Biochem., 273: 221-228 (1999)(two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β₂-microglobulin, ribosomal RNA, and the like. Techniques for quantitative PCR are well-known to those of ordinary skill in the art, as exemplified in the following references that are incorporated by reference: Freeman et al, Biotechniques, 26; 112-126 (1999); Becker-Andre et al, Nucleic Acids Research, 17: 9437-9447 (1989); Zimmerman et al, Biotechniques, 21: 268-279 (1996); Diviacco et al, Gene, 122: 3013-3020 (1992); Becker-Andre et al, Nucleic Acids Research, 17: 9437-9446 (1989); and the like.

“Polynucleotide” or “oligonucleotide” are used interchangeably and each mean a linear polymer of natural or modified nucleotide monomers. Monomers making up polynucleotides and oligonucleotides include deoxyribonucleotides, ribonucleotides, 2′-deoxy-3′-phosphorothioate nucleosides, peptide nucleic acids (PNAs), and the like, that are capable of specifically binding to a natural polynucleotide by way of a regular pattern of monomer-to-monomer interactions, such as Watson-Crick type of base pairing, base stacking, Hoogsteen or reverse Hoogsteen types of base pairing, or the like. Polynucleotides typically range in size from a few monomeric units, e.g. 5-40, when they are usually referred to as “oligonucleotides,” to several thousand monomeric units. Whenever a polynucleotide is represented by a sequence of letters (upper or lower case), such as “ATGCCTG,” it will be understood that the nucleotides are in 5′→3′order from left to right and that “A” denotes deoxyadenosine, “C” denotes deoxycytidine, “G” denotes deoxyguanosine, and “T” denotes thymidine, “1” denotes deoxyinosine, “U” denotes uridine. Unless otherwise noted the terminology and atom numbering conventions will follow those disclosed in Strachan and Read, Human Molecular Genetics 2 (Wiley-Liss, New York, 1999). Usually polynucleotides comprise the four natural nucleosides (e.g. deoxyadenosine, deoxycytidine, deoxyguanosine, deoxythymidine for DNA) linked by phosphodiester linkages; however, they may also comprise non-natural nucleotide analogs, e.g. including modified bases, sugars, or internucleosidic linkages. It is clear to those skilled in the art when oligonucleotides having natural or non-natural nucleotides may be employed, e.g. where processing by enzymes is called for, usually polynucleolides consisting solely of natural nucleotides are required. Likewise, where an enzyme has specific oligonucleotide or polynucleotide substrate requirements for activity, e.g. single stranded DNA, RNA/DNA duplex, or the like, then selection of appropriate composition for the oligonucleotide or polynucleotide substrates is well within the knowledge of one of ordinary skill, especially with guidance from treatises, such as Sambrook et al, Molecular Cloning, Second Edition (Cold Spring Harbor Laboratory, New York, 1989), and like references.

“Primer” means an oligonucleotide, either natural or synthetic that is capable, upon forming a duplex with a polynucleotide template of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following references that are incorporated by reference: Dieffenbach, editor, PCR Primer: A Laboratory Manual, 2^{nd} Edition (Cold Spring Harbor Press, New York, 2003).

“Quality score” means a measure of the probability that a base assignment at a particular sequence location is correct. A variety methods are well known to those of ordinary skill for calculating quality scores for particular circumstances, such as, for bases called as a result of different sequencing chemistries, detection systems, base-calling algorithms, and so on. Generally, quality score values are monotonically related to probabilities of correct base calling. For example, a quality score, or Q, of 10 may mean that there is a 90 percent chance that a base is called correctly, a Q of 20 may mean that there is a 99 percent chance that a base is called correctly, and so on. For some sequencing platforms, particularly those using sequencing-by-synthesis chemistries, average quality scores decrease as a function of sequence read length, so that quality scores at the beginning of a sequence read are higher than those at the end of a sequence read, such declines being due to phenomena such as incomplete extensions, carry forward extensions, loss of template, loss of polymerase, capping failures, deprotection failures, and the like.

“Sequence read” means a sequence of nucleotides determined from a sequence or stream of data generated by a sequencing technique, which determination is made, for example, by means of base-calling software associated with the technique, e.g. base-calling software from a commercial provider of a DNA sequencing platform. A sequence read usually includes quality scores for each nucleotide in the sequence. Typically, sequence reads are made by extending a primer along a template nucleic acid, e.g. with a DNA polymerase or a DNA lipase. Data is generated by recording signals, such as optical, chemical (e.g. pH change), or electrical signals, associated with such extension. Such initial data is converted into a sequence read.

“Sequence tag” (or “tag”) or “barcode” means an oligonucleotide that is attached to a polynucleotide or template molecule and is used to identify and/or track the polynucleotide or template in a reaction or a series of reactions. A sequence tag may be attached to the 3′- or 5′-end of a polynucleotide or template or it may be inserted into the interior of such polynucleotide or template to form a linear conjugate, sometime referred to herein as a “tagged polynucleotide,” or “tagged template,” or “tag-polynucleotide conjugate,” “tag-molecule conjugate,” or the like. Sequence tags may vary widely in size and compositions; the following references, which are incorporated herein by reference, provide guidance for selecting sets of sequence tags appropriate for particular embodiments: Brenner, U.S. Pat. No. 5,635,400; Brenner and Macevicz, U.S. Pat. No. 7,537,897; Brenner et al, Prot. Natl. Acad. Sci., 97: 1665-1670 (2000); Church et al, European patent publication 0 303 459; Shoemaker et al, Nature Genetics 14: 450-456 (1996); Morris et al, European patent publication 0799897A1; Wallace, U.S. Pat. No. 5,981,179; and the like. Lengths and compositions of sequence tags can vary widely, and the selection of particular lengths and/or compositions depends on several factors including, without limitation, how tags are used to generate a readout, e.g. via a hybridization reaction or via an enzymatic reaction, such as sequencing; whether they are labeled, e.g. with a fluorescent dye or the like; the number of distinguishable oligonucleotide tags required to unambiguously identify a set of polynucleotides, and the like, and how different must tags of a set be in order to ensure reliable identification, e.g. freedom from cross hybridization or misidentification from sequencing errors. In one aspect, sequence tags can each have a length within a range of from 2 to 36 nucleotides, or from 4 to 30 nucleotides, or from 8 to 20 nucleotides, or from 6 to 10 nucleotides, respectively. In one aspect, sets of sequence tags are used wherein each sequence tag of a set has a unique nucleotide sequence that differs from that of every other tag of the same set by at least two bases; in another aspect, sets of sequence tags are used wherein the sequence of each tag of a set differs from that of every other tag of the same set by at least three bases.

What is claimed is:

1. A method for sequencing nucleic acids comprising:
preparing DNA templates front nucleic acids in a sample;
labeling by sampling the DNA templates to form a multiplicity tag-template conjugates, wherein substantially every DNA template of a tag-template conjugate has a unique mosaic tag comprising alternating constant regions and variable regions, each constant region having a position in the mosaic tag and a length of from 1 to 10 nucleotides of a predetermined sequence and each variable region having a position in the mosaic tag and a length of from 1 to 10 randomly selected nucleotides, such that constant regions having the same positions have the same lengths and variable region having the same positions have the same lengths;
amplifying the multiplicity of tag-template conjugates;
generating a plurality of sequence reads for each of the amplified tag-template conjugates; and
determining a nucleotide sequence of each of the nucleic acids by determining a consensus nucleotide at each nucleotide position of each plurality of sequence reads having identical mosaic tags.

2. The method of claim 1 wherein said mosaic tag has a length in the range of from 10 to 100 nucleotides.

3. The method of claim 2 wherein said mosaic tag comprises at least 8 nucleotide positions with randomly selected nucleotides.

4. The method of claim 1 wherein said sample is from a species and wherein said predetermined sequences of said constant regions of said mosaic tag are selected so that nonspecific hybridization by said predetermined sequences to genomic sequences of the species or products thereof is minimized.

5. The method of claim 4 wherein said sample is from a human.

6. A method of determining clonotypes of an immune repertoire, the method comprising the steps:
(a) obtaining a sample from an individual comprising T-cells and/or B-cells;
(b) attaching mosaic tags to molecules of recombined nucleid acids of T-cell receptor genes or immunoglobulin genes of the T-cells and/or B-cells to form tag-molecule conjugates, wherein substantially every molecule of the tag-molecule conjugates has a unique mosaic tag;
(c) amplifying the tag-molecule conjugates;
(d) sequencing the tag-molecule conjugates; and
(e) aligning sequence reads of like mosaic tags to determine sequence reads corresponding to the same clonotypes types the repertoire.

7. The method of claim 6 wherein said step of aligning further includes determining a nucleotide sequence of each of said clonotypes of each of said tag-molecule conjugate by determining a majority nucleotide at each nucleotide position of said clonotypes of said like mosaic tags.

8. The method of claim 6 wherein said step of attaching includes labeling by sampling said molecules of recombined nucleic acids.

9. A method of determining clonotypes of an immune repertoire, the method comprising the steps:
(a) obtaining a sample from an individual comprising T-cells and/or B-cells;
(b) labeling by sampling molecules from the T-cells and/or B-cells to form tag-molecule conjugates, wherein each tag of said conjugates has a sequence of the form:
[(N₁N₂ . . . N_{Kj})(b₁b₂ . . . b_{Lj})]M
wherein each Nᵢ, for i=1, 2, . . . , Kⱼ, is a nucleotide randomly selected from the group consisting of A, C, G and T; Kᵢ is an integer in the range of from 1 to 10 for each j less than or equal to M; each bᵢ, for i=1, 2, . . . Lⱼ, is a nucleotide; Lⱼ is an integer in the range of from 1 to 10 for each j less than or equal to M; such that every sequence tag (i) has the same Kj for every j and (ii) has the same sequences b₁b₂ . . . b_{Lj} for every j; and M is an integer greater than or equal to 2; and each molecule of said conjugates comprises a recombined nucleic acid from a T-cell receptor gene or an immunoglobulin gene;
(c) sequencing the tag-molecule conjugates; and
(d) aligning sequence reads of like tags to determine like clonotypes.

10. The method of claim 9 wherein said step of aligning further includes determining a nucleotide sequence of each of said clonotype of each of said tag-molecule conjugate by determining a majority nucleotide at each nucleotide position of said clonotypes of said like tags.

11. The method of claim 10 wherein said step of attaching is implemented in a reaction mixture such that said tags are present in the reaction mixture in a concentration at least 100 time that of said molecules of recombined nucleic acid.


SEQUENCE LISTING (11 sequences)

SEQ ID NO: 1 - DNA, 24 bp, Artificial Sequence
  primer
agttctggctaacctgtagagcca

SEQ ID NO: 2 - DNA, 24 bp, Artificial Sequence
  primer
agttcgggctaacctgtcgagcca

SEQ ID NO: 3 - DNA, 24 bp, Artificial Sequence
  primer
agttccggctaacctgtcgagcca

SEQ ID NO: 4 - DNA, 22 bp, Artificial Sequence
  primer
nnnnnnnnnnnnnnnnnnnnnn

SEQ ID NO: 5 - DNA, 16 bp, Artificial Sequence
  probe
nnnnnnnnnnnnnnnn

SEQ ID NO: 6 - DNA, 30 bp, Artificial Sequence
  probe or tag
nnctnntgnngtnngcnntgnngtnntann

SEQ ID NO: 7 - DNA, 30 bp, Artificial Sequence
  probe or tag
nnctntgnnngtngcntgnnngtnntannn

SEQ ID NO: 8 - DNA, 65 bp, Artificial Sequence
  probe or tag
acgagcctcatgcgtagannctntnnacnngtncnnacnngtnnnctcacctgaggagacggtga

SEQ ID NO: 9 - DNA, 65 bp, Artificial Sequence
  probe or tag
acgagcctcatgcgtagannctntnnacnngtncnnacnngtnnnctcacctgaggagacagtga

SEQ ID NO: 10 - DNA, 65 bp, Artificial Sequence
  probe or tag
acgagcctcatgcgtagannctntnnacnngtncnnacnngtnnncttacctgaagagacggtga

SEQ ID NO: 11 - DNA, 65 bp, Artificial Sequence
  probe or tag
acgagcctcatgcgtagannctntnnacnngtncnnacnngtnnncttacctgaggagacggtga